(19) United States
(12) Patent Application Publication
(10) Pub. No.: US 2023/0241349 A1
(43) Pub. Date: Aug. 3, 2023

(54) ELONGATED CONVEYANCE ASSEMBLY HAS TACTILE FEATURE

(71) Applicant: Boston Scientific Medical Device Limited, Galway (IE)

(72) Inventor: Matthew DiCicco, Guelph (CA)

(21) Appl. No.: 18/192,960

(22) Filed: Mar. 30, 2023

Related U.S. Application Data
(63) Continuation of application No. PCT/IB2021/057919, filed on Aug. 30, 2021.
(60) Provisional application No. 63/085,256, filed on Sep. 30, 2020.

(51) Int. Cl. A61M 25/00; A61M 25/06; A61M 25/10

(52) U.S. Cl. CPC A61M 25/0068; A61M 25/0662; A61M 25/1025; A61M 2025/0681; A61M 2025/1061; A61M 2025/1084; A61M 2029/025

(57) ABSTRACT

An apparatus for use with an elongated medical device configured to be tacitly contacted by a user is disclosed. The apparatus includes an elongated medical-conveyance assembly defining an elongated conveyance lumen configured to receive the elongated medical device. A tactile feature is located at (in) the elongated conveyance lumen. The elongated guidewire-conveyance assembly may include biocompatible material properties suitable for sufficient performance.

### CROSS-REFERENCE TO RELATED APPLICATIONS

This application is a continuation of and claims the benefit of International Application Number PCT/IB2021/057919, entitled “ELONGATED CONVEYANCE ASSEMBLY HAS TACTILE FEATURE,” and filed Aug. 30, 2021, which claims the benefit of U.S. Provisional Application No. 63/085,256, entitled “ELONGATED CONVEYANCE ASSEMBLY HAS TACTILE FEATURE,” and filed Sep. 30, 2020, which are hereby incorporated by reference in their entireties.

### TECHNICAL FIELD

This document relates to the technical field of (and is not limited to) (A) an elongated medical-conveyance assembly having a tactile feature (and method therefor), and (B) an elongated guidewire-conveyance assembly having a tactile feature (and method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with existing elongated guidewire-conveyance assemblies and guidewires (also called the existing technology). After much study of, and experimentation with, the existing elongated medical guidewire-conveyance assemblies and guidewires, an understanding (at least in part) of the problem and its solution have been identified (at least in part) and are articulated (at least in part) as follows:

During use of exchange-length guidewires in catheterization procedures, it may be difficult for physicians to know how far a guidewire is inserted into a sheath assembly and/or a dilator assembly; this is done without direct cues (e.g. depth markers) but may be indirectly visualized (if desired) through catheter-imaging techniques (such as, medical imaging systems, fluoroscopy systems, echo systems, etc., and any equivalent thereof). This may apply to back-loading guidewires through a sheath assembly and/or a dilator assembly already in place in the vasculature (within the patient) or advancing the sheath assembly and/or the dilator assembly over already vascularized guidewires, etc.

With the advent of exchange-length radiofrequency guidewires and mechanical puncture wires used in transseptal procedures, there is, at present, no physical or tactile way (assurance) for physicians to know (detect) when a distal tip of the guidewire is aligned with a distal tip of a dilator assembly when compared to traditional toolsets. It will be appreciated that the physician may indirectly visualize the position with catheter-imaging techniques (if desired), however, this case may add additional burden to the procedure in comparison to the traditional toolsets described. In using the NRG (TRADEMARK) Transseptal Needle (manufactured by the BAYLIS MEDICAL COMPANY headquarter in Canada), for example, this physical (or tactile) reassurance may be provided by the length of the needle matching the length of the sheath assembly and/or the dilator assembly. The physician may be able to use the position of the NRG handle in reference to the sheath hub to delineate the relative distance of the distal tips from each other; this may be done, preferably or advantageously (if desired), without using a medical-imaging system (such as, fluoroscopy, echo, an electroanatomical mapping system, etc.).

A way to passively detect when the distal end sections of a guidewire and a dilator are aligned may be important to (A) streamline procedural workflow before formation of a puncture; (B) reduce reliance on, or need for, a medical imaging system; and/or (C) prevent potential for inadvertent puncture or vasculature damage from over-advancement through the sheath assembly and/or the dilator assembly.

To mitigate, at least in part, at least one problem associated with the existing technology, there is provided (in accordance with a major aspect) an apparatus. The apparatus is for use with (is configured to be used with) an elongated medical device configured to be tacitly contacted by a user. The apparatus includes and is not limited to (comprises) an elongated medical-conveyance assembly defining an elongated conveyance lumen configured to receive the elongated medical device. A tactile feature is located at (in) the elongated conveyance lumen. In accordance with an option (and not limited thereto), the elongated medical-conveyance assembly includes an elongated guidewire-conveyance assembly having a distal section and a proximal section, with the elongated conveyance lumen extending between the distal section and the proximal section, and with the elongated conveyance lumen configured to receive the elongated medical device. The elongated medical device includes an elongated guidewire assembly.

To mitigate, at least in part, at least one problem associated with the existing technology, there is provided (in accordance with a major aspect) an apparatus. The apparatus is for use with (is configured to be used with) an elongated guidewire assembly having a guidewire distal tip section and a guidewire proximal section configured to be tacitly contacted by a user. The apparatus includes and is not limited to (comprises) an elongated guidewire-conveyance assembly having a distal section and a proximal section. An elongated conveyance lumen extends between the distal section and the proximal section. The elongated conveyance lumen is configured to receive the guidewire distal tip section of the elongated guidewire assembly from the proximal section. A tactile feature is located in (at) the elongated conveyance lumen.

To mitigate, at least in part, at least one problem associated with the existing technology, there is provided (in accordance with a major aspect) a method. The method is for (is associated with) using an elongated medical device configured to be contacted by a user. The method includes and is not limited to (comprises) receiving the elongated medical device into an elongated conveyance lumen of an elongated medical-conveyance assembly. The method also includes moving the elongated medical device along the elongated conveyance lumen toward a tactile feature located at the elongated conveyance lumen.

To mitigate, at least in part, at least one problem associated with the existing technology, there is provided (in accordance with a major aspect) a method. The method is for (is associated with) using an elongated guidewire assembly having a guidewire distal tip section and a guidewire proximal section configured to be contacted by a user. The method includes and is not limited to (comprises) receiving the guidewire distal tip section of the elongated guidewire assembly into an elongated conveyance lumen of an elongated guidewire-conveyance assembly having a distal section and a proximal section, with the elongated conveyance lumen extending between the distal section and the proximal section. The method also includes moving the guidewire distal tip section along the elongated conveyance lumen toward a tactile feature located in the elongated conveyance lumen. In accordance with an option (and not limited thereto), the method may also include interacting the guidewire distal tip section of the elongated guidewire assembly with the tactile feature; this is done, preferably, in such a way that the tactile feature, in use, imparts a tactile vibration to the guidewire distal tip section, and the tactile vibration (in use) travels along the elongated guidewire assembly and toward the guidewire proximal section and then to the user contacting the guidewire proximal section in response to contact between the guidewire distal tip section and the tactile feature.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 and FIG. 2 depict a front perspective view (FIG. 1) and a cross-sectional view (FIG. 2) of embodiments (implementations) of an elongated guidewire-conveyance assembly having a tactile feature; and

FIG. 3 and FIG. 4 depict a cross-sectional view (FIG. 3) and a side view (FIG. 4) of embodiments (implementations) of the elongated guidewire-conveyance assembly and the tactile feature of FIG. 1; and

FIG. 5 and FIG. 6 depict a front perspective partial section view (FIG. 5) and a close-up perspective view (FIG. 6) of embodiments (implementations) of the elongated guidewire-conveyance assembly and the tactile feature of FIG. 1; and

FIG. 7 and FIG. 8 depict a side view (FIG. 7) and a cross-sectional view (FIG. 8) of embodiments (implementations) of the elongated guidewire-conveyance assembly and the tactile feature of FIG. 1; and

FIG. 9 and FIG. 10 depict cross-sectional views of embodiments (implementations) of the elongated guidewire-conveyance assembly and the tactile feature of FIG. 1; and

FIG. 11 and FIG. 12 depict cross-sectional views of embodiments (implementations) of the elongated guidewire-conveyance assembly and the tactile feature of FIG. 1; and

FIG. 13 and FIG. 14 depict cross-sectional views of embodiments (implementations) of the elongated guidewire-conveyance assembly and the tactile feature of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

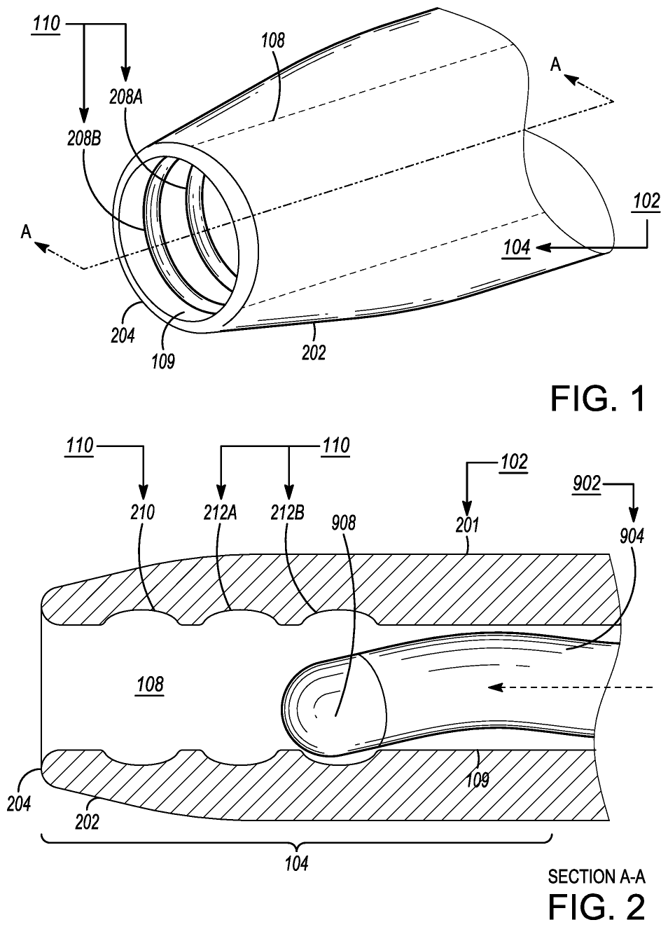

FIG. 1 and FIG. 2 depict a front perspective view (FIG. 1) and a cross-sectional view (FIG. 2) of embodiments (implementations) of an elongated guidewire-conveyance assembly 102 having a tactile feature 110. The cross-sectional view of FIG. 2 is taken along a cross-sectional line A-A of FIG. 1. It will be appreciated that the definition of tactile feature 110 is any element configured to be responsive to sensed or sensory stimuli.

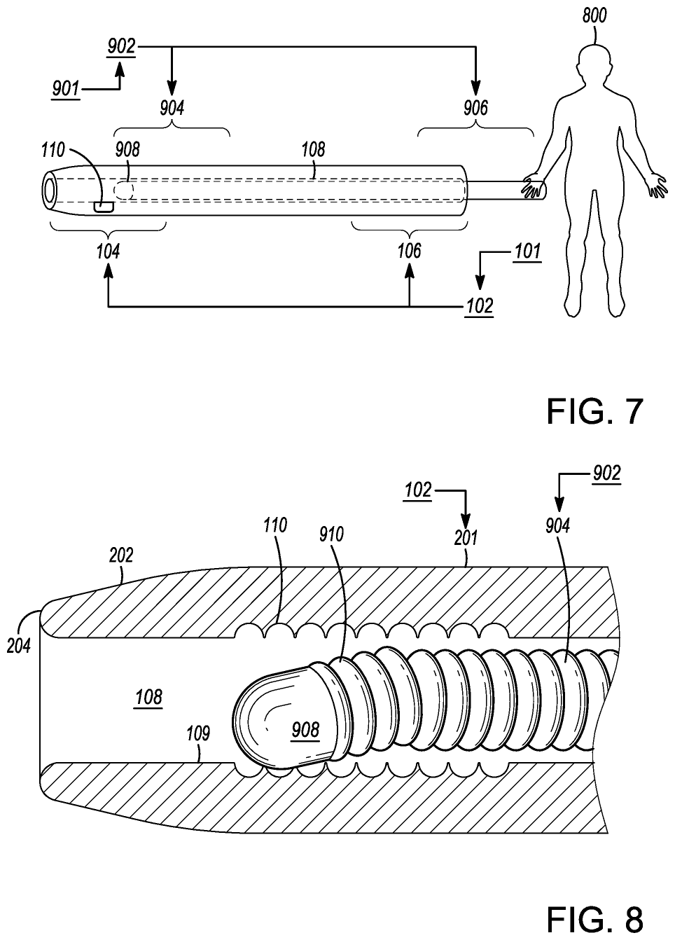

Referring to the embodiment (implementation) as depicted in FIG. 1, the elongated guidewire-conveyance assembly 102 includes a distal section 104. The elongated guidewire-conveyance assembly 102 defines an elongated conveyance lumen 108. A lumen surface 109 (internal lumen surface) faces the interior of the elongated conveyance lumen 108 (of the elongated guidewire-conveyance assembly 102). The distal section 104 includes (preferably) a distal tapered section 202 and a distal tip 204 extending from the distal tapered section 202. The tactile feature 110 is located in the elongated conveyance lumen 108. The tactile feature 110 is (preferably) located on the lumen surface 109. For instance, the tactile feature 110 may include a thick recessed protrusion 208A positioned in the elongated conveyance lumen 108. For instance, the tactile feature 110 may include a thin recessed protrusion 208B positioned in the elongated conveyance lumen 108. The tactile feature 110 may, for instance, include at least one (or more) raised features (such as, at least one raised feature, a relief feature), at least one (or more) recessed features, at least one or more speed bumps, etc. It will be appreciated that a relief feature is a feature that may be raised from a baseline surface or a feature that may be lowered from the baseline surface, etc. The tactile feature 110 is, preferably, formed on the lumen surface 109. The tactile feature 110 is positioned at the distal section 104 of the elongated guidewire-conveyance assembly 102. The tactile feature 110 is configured to interact with (contact) a guidewire distal tip section 904 (of an elongated guidewire assembly 902) in response to the guidewire distal tip section 904 making (physical) contact with the tactile feature 110. The tactile feature 110 is configured to provide tactile feedback to the user of the guidewire distal tip section 904. The tactile feature 110 may include bumps, divots, cuts, dimples, etc. configured to tactilely interact with the guidewire distal tip section 904. Once contact is made, vibrations are imparted from the tactile feature 110 to the guidewire distal tip section 904 and then to the user making contact with the elongated guidewire assembly 902 (better depicted in FIG. 7). Returning to FIG. 1, the interaction between the tactile feature 110 and the guidewire distal tip section 904 allow the physician (user) to tactilely sense (feel) when the guidewire distal tip section 904 has reached the tactile feature 110 during urged movement (advancement or retraction) of the guidewire distal tip section 904 relative to the tactile feature 110.

Referring to the embodiment (implementation) as depicted in FIG. 1, the elongated guidewire-conveyance assembly 102 is configured to be inserted into a confined space defined by a living body (the patient).

Referring to the embodiment (implementation) as depicted in FIG. 1, the elongated guidewire-conveyance assembly 102 includes biocompatible material properties suitable for sufficient performance (such as, electric dielectric strength, thermal performance, electric insulation, corrosion, water resistance, heat resistance) for compliance with industrial and regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiment (implementation) as depicted in FIG. 1, the elongated conveyance lumen 108 is configured to receive and contain the guidewire distal tip section 904 (of the elongated guidewire assembly 902). The tactile feature 110 may include a series of raised or recessed features aligned along a longitudinal length of the elongated conveyance lumen 108. The tactile features 110 (such as, raised features and/or recessed features, etc., and any equivalent thereof) may be large enough that they may be tacitly felt while the guidewire distal tip section 904 (of the elongated guidewire assembly 902) contacts (interacts) with them. The tactile feature 110 is configured to facilitate tactile sensing of the location of the guidewire distal tip section 904. The tactile feature 110 may be located at the guidewire distal tip section 904 and/or at the proximal section of the elongated guidewire assembly 902 (if so desired). The guidewire distal tip section 904 (of the elongated guidewire assembly 902) is configured to be able to traverse through the elongated conveyance lumen 108 of the elongated guidewire-conveyance assembly 102. The tactile feature 110 is, preferably, configured to avoid (has a material for avoiding) wearing out the guidewire distal tip section 904. It will be appreciated that it is not necessary for the guidewire distal tip section 904 to be especially designed to interact with the tactile feature 110. In accordance with an option, the tactile feature 110 is, preferably, configured to avoid (has a material for avoiding) wear down as a result from interaction with the guidewire.

Referring to the embodiment (implementation) as depicted in FIG. 2, the tactile feature 110 is configured to interact with the guidewire distal tip section 904 (of the elongated guidewire assembly 902) in response to movement of the guidewire distal tip section 904 along the distal section 104. The elongated guidewire-conveyance assembly 102 includes a shaft portion 201. The tactile feature 110 includes a distal recess 210. The tactile feature 110 includes grouped recesses (212A, 212B).

Referring to the embodiment (implementation) as depicted in FIG. 2, the guidewire distal tip section 904 includes a distal puncture device 908. The distal puncture device 908 (or the elongated guidewire assembly 902) may include (and is/are not limited to) a radiofrequency puncture device, such as the BAYLIS (TRADEMARK) POWERWIRE (REGISTERED TRADEMARK) radio frequency guidewire manufactured by BAYLIS MEDICAL COMPANY (headquartered in Canada). In accordance with another embodiment, the distal puncture device 908 may, for instance, include an elongated guidewire having a distal tip section presenting a mechanical cutting portion (for this case, a puncture site is formed by physically moving the mechanical cutting portion into the biological feature).

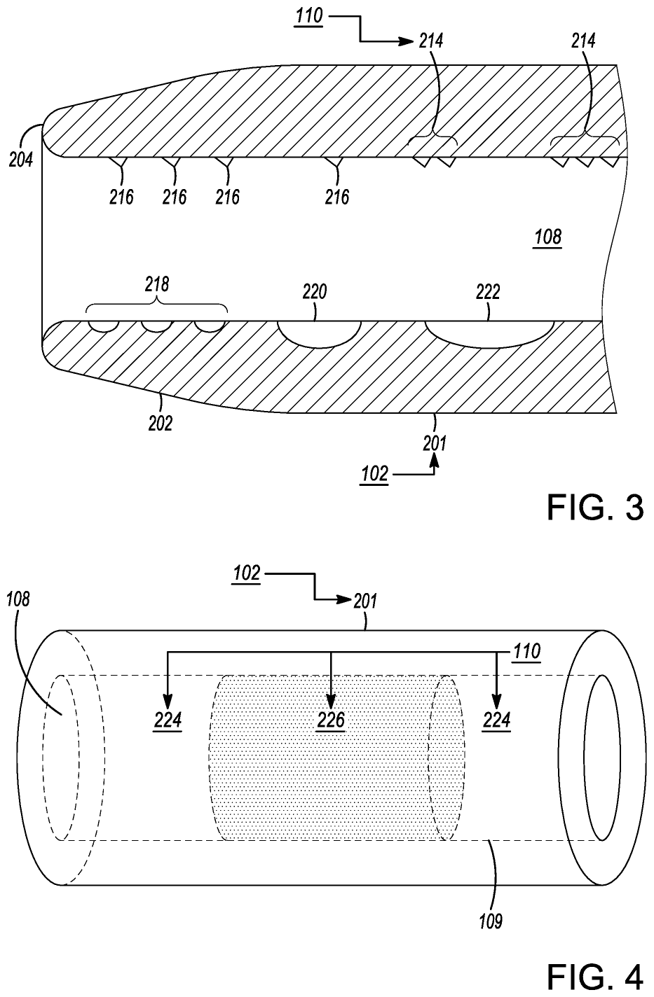

FIG. 3 and FIG. 4 depict a cross-sectional view (FIG. 3) and a schematic side view (FIG. 4) of embodiments (implementations) of the elongated guidewire-conveyance assembly 102 and the tactile feature 110 of FIG. 1. The cross-sectional view of FIG. 3 is taken along a cross-sectional line A-A of FIG. 1.

Referring to the embodiment (implementation) as depicted in FIG. 3, the tactile feature 110 may include at least one or more raised features formed on the lumen surface 109. The tactile feature 110 may include at least one or more recessed features formed on the lumen surface 109. The tactile feature 110 may include at least one or more raised features and at least one or more recessed features (both of which are formed on the lumen surface 109). It will be appreciated that the spacing between the multiple instances of the tactile feature 110 may be spaced relatively closer or further far apart from each other, in close succession or more gradually spaced, etc. The tactile feature 110 may be placed at the distal section 104 (of the elongated guidewire-conveyance assembly 102), a proximal end (of the elongated guidewire-conveyance assembly 102, as depicted in FIG. 7), or at a position located in between, etc.

Referring to the embodiment (implementation) as depicted in FIG. 3, the tactile feature 110 may include different styles of features (raised and/or recessed features). For instance, the tactile feature 110 may include features grouped or spaced as needed to convey intended information to the user. The elongated guidewire-conveyance assembly 102 includes a shaft portion 201 with a distal tapered section 202 extending from the shaft portion 201. The tactile feature 110 may include a grouping of spaced-apart raised markers 214 (that extend, preferably into the interior of the lumen surface 109). The tactile feature 110 may include a single raised marker 216 (that extends, preferably into the interior of the lumen surface 109). The tactile feature 110 may include a grouping of recessed markers 218 (that extend, preferably into the shaft portion 201). The tactile feature 110 may include a medium-sized recessed marker 220. The tactile feature 110 may include a larger-sized recess marker 222.

Referring to the embodiment (implementation) as depicted in FIG. 4, the tactile feature 110 includes (preferably) sections of varying surface roughness formed on the lumen surface 109. The tactile feature 110 includes (preferably) a smooth tactile lumen section 224 and a rough tactile lumen section 226 positioned next to the smooth tactile lumen section 224. The tactile feature 110 may include the smooth tactile lumen section 224 and the rough tactile lumen section 226 positioned in an alternating arrangement.

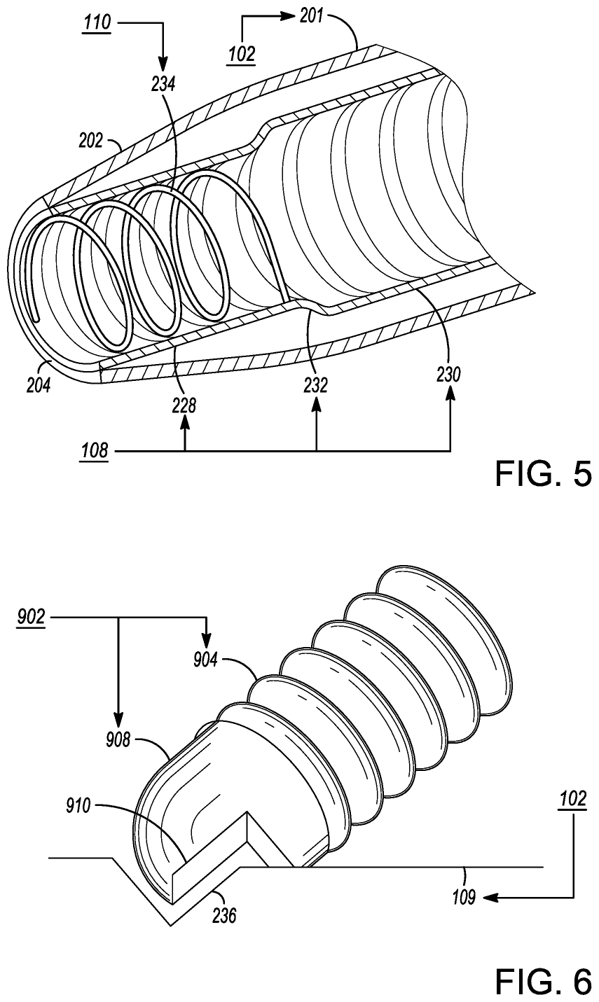

FIG. 5 and FIG. 6 depict a front perspective partial section view (FIG. 5) and a close-up perspective view (FIG. 6) of embodiments (implementations) of the elongated guidewire-conveyance assembly 102 and the tactile feature 110 of FIG. 1.

Referring to the embodiment (implementation) as depicted in FIG. 5, the tactile feature 110 includes a helically-formed marker 234 (a relief pattern that may be either raised or recessed). The helically-formed marker 234 may be continuous if so desired. The elongated conveyance lumen 108 may include a distal lumen section 228, a proximal lumen section 230 positioned adjacently to the distal lumen section 228, and a transition lumen section 232 positioned adjacently to the proximal lumen section 230. The distal lumen section 228 may have a smooth inner surface. The proximal lumen section 230 may have a smooth inner surface. The transition lumen section 232 may have a smooth inner surface.

Referring to the embodiment (implementation) as depicted in FIG. 6, the tactile feature 110 includes a surface feature 236 formed on the lumen surface 109. The guidewire distal tip section 904 (or the distal puncture device 908) includes a guidewire interference feature 910. The surface feature 236 and the guidewire interference feature 910 are configured to interact with each other.

FIG. 7 and FIG. 8 depict a side view (FIG. 7) and a cross-sectional view (FIG. 8) of embodiments (implementations) of the elongated guidewire-conveyance assembly 102 and the tactile feature 110 of FIG. 1. The cross-sectional view of FIG. 8 is taken along a cross-sectional line A-A of FIG. 1.

Referring to the embodiment (implementation) as depicted in FIG. 7, the elongated medical-conveyance assembly 101 is configured for use with an elongated medical device 901 configured to be tacitly contacted by a user 800. The elongated medical-conveyance assembly 101 is configured to be inserted into a confined space defined by a living body (the patient). The elongated medical-conveyance assembly 101 may include (and is not limited to) an elongated catheter assembly, an elongated dilator assembly or an elongated sheath assembly, and any equivalent thereof. The elongated catheter assembly includes a flexible tube configured to be inserted through a narrow opening into a body cavity. The elongated dilator assembly is configured to induce dilation, that is, to expand an opening or passage through a biological wall or feature. The elongated sheath assembly is configured to convey (pass) a medical device, such as an obturator, a cutting instrument, etc.

Referring to the embodiment (implementation) as depicted in FIG. 7, the tactile feature 110 may be utilized for (or applied to) interactions between a first elongated medical device (such as a sheath assembly) and a second elongated medical device (such as a dilator assembly). The tactile feature 110 may be utilized for (or applied to) a catheter-in-catheter configuration, a pigtail catheters, a micro-catheter, etc., and any equivalent thereof. For instance, for application with endovascular requirements, the tactile feature 110 may be applied to catheter-in-catheter configurations where the distal ends of the catheters are shaped and fluoro usage (medical imaging) is relatively higher. The tactile feature 110 may be utilized for any other procedure that may require use of a guidewire through an introducer device (such as for an epicardial procedure). For the case where the elongated guidewire-conveyance assembly 102 includes an elongated medical dilator assembly, the tactile feature 110 (raised/recessed features) may be molded in during a dilator tipping process with a specialized tipping mandrel, or the tactile feature 110 may be machined into the lumen after molding, etc.

Referring to the embodiment (implementation) as depicted in FIG. 7, the elongated medical-conveyance assembly 101 defines the elongated conveyance lumen 108 configured to receive the elongated medical device 901. The tactile feature 110 is located in (at) the elongated conveyance lumen 108. In accordance with an option (and not limited thereto), the elongated medical-conveyance assembly 101 includes the elongated guidewire-conveyance assembly 102. The elongated guidewire-conveyance assembly 102 has the distal section 104 and the proximal section 106, with the elongated conveyance lumen 108 extending between the distal section 104 and the proximal section 106. The elongated conveyance lumen 108 is configured to receive the elongated medical device 901. The elongated medical device 901 includes an elongated guidewire assembly 902.

Referring to the embodiment (implementation) as depicted in FIG. 7, the elongated guidewire-conveyance assembly 102 is configured to be used with the elongated guidewire assembly 902 having the guidewire distal tip section 904 and the guidewire proximal section 906 configured to be tacitly contacted by the user 800. The elongated guidewire-conveyance assembly 102 has the distal section 104 and the proximal section 106. The elongated conveyance lumen 108 extends between the distal section 104 and the proximal section 106. The elongated conveyance lumen 108 is configured to receive the guidewire distal tip section 904 of the elongated guidewire assembly 902 from the proximal section 106. A tactile feature 110 is located in (at) the elongated conveyance lumen 108. In accordance with an option (and not limited thereto), the tactile feature 110 extends from the distal section 104 toward an interior of the elongated conveyance lumen 108.

Referring to the embodiment (implementation) as depicted in FIG. 7, the tactile feature 110 is configured to tacitly interact with the guidewire distal tip section 904 (of the elongated guidewire assembly 902); this is done, preferably, in response to contact being made between the guidewire distal tip section 904 and the tactile feature 110 while the guidewire distal tip section 904 is moved past the tactile feature 110. The tactile feature 110 is (preferably) configured to tacitly interact with the guidewire distal tip section 904; this is done (preferably) in such a way that the tactile feature 110, in use, imparts a tactile vibration into the guidewire distal tip section 904, and the tactile vibration (in use) travels along the elongated guidewire assembly 902 toward the guidewire proximal section 906 and then to the user who tacitly contacts the guidewire proximal section 906 in response to tactile contact between the guidewire distal tip section 904 and the tactile feature 110. The tactile feature 110 is (preferably) configured to tacitly interact with the guidewire distal tip section 904; this is done, preferably, in response to contact being made between the guidewire distal tip section 904 and the tactile feature 110 while the guidewire distal tip section 904 is moved past the tactile feature 110. The tactile feature 110 is (preferably) configured to tacitly interact with the guidewire distal tip section 904; this is done (preferably) in such a way that the tactile feature 110, in use, imparts a tactile vibration into the guidewire distal tip section 904, and the tactile vibration (in use) travels along the elongated guidewire assembly 902 toward the guidewire proximal section 906 and then to the user who tacitly contacts the guidewire proximal section 906 in response to tactile contact between the guidewire distal tip section 904 and the tactile feature 110.

Referring to the embodiment (implementation) as depicted in FIG. 7, there is depicted a method for using the elongated medical device 901 (configured to be contacted by a user 800). The method includes receiving the elongated medical device 901 into an elongated conveyance lumen 108 of an elongated medical-conveyance assembly 101. The method also includes moving the elongated medical device 901 along the elongated conveyance lumen 108 toward a tactile feature 110 located at the elongated conveyance lumen 108.

Referring to the embodiment (implementation) as depicted in FIG. 7, there is depicted a method for using the elongated guidewire assembly 902 (having the guidewire distal tip section 904 and a guidewire proximal section 906 configured to be contacted by the hand or finger of the user 800). The method includes receiving the guidewire distal tip section 904 of the elongated guidewire assembly 902 into an elongated conveyance lumen 108 of an elongated guidewire-conveyance assembly 102 having a distal section 104 and a proximal section 106, with the elongated conveyance lumen 108 extending between the distal section 104 and the proximal section 106. The method also includes moving the guidewire distal tip section 904 along the elongated conveyance lumen 108 toward a tactile feature 110 located in the elongated conveyance lumen 108. The method preferably includes interacting (urging the interaction of) the guidewire distal tip section 904 of the elongated guidewire assembly 902 with the tactile feature 110; this is done (preferably) in such a way that the tactile feature 110, in use, imparts a tactile vibration to the guidewire distal tip section 904, and the tactile vibration (in use) travels along the elongated guidewire assembly 902 toward the guidewire proximal section 906 and then to the user the contacts the guidewire proximal section 906 in response to contact between the guidewire distal tip section 904 and the tactile feature 110.

Referring to the embodiment (implementation) as depicted in FIG. 8, the tactile feature 110 is configured to interact with a guidewire interference feature 910 of the guidewire distal tip section 904 (or the distal puncture device 908). The guidewire interference feature 910 is positioned on an outer surface of the guidewire distal tip section 904 (or, generally, the elongated guidewire assembly 902).

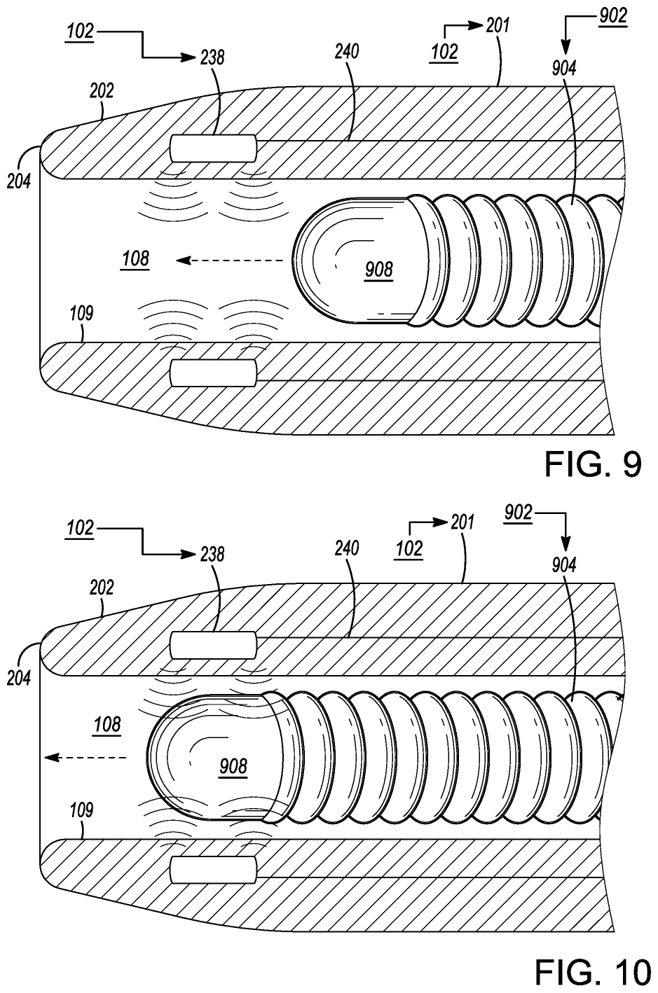

FIG. 9 and FIG. 10 depict cross-sectional views of embodiments (implementations) of the elongated guidewire-conveyance assembly 102 and the tactile feature 110 of FIG. 1. The cross-sectional views of FIG. 9 and FIG. 10 are taken along a cross-sectional line A-A of FIG. 1.

Referring to the embodiments (implementations) as depicted in FIG. 9 and FIG. 10, the elongated guidewire-conveyance assembly 102 includes the shaft portion 201. The shaft portion 201 defines a recess feature 250 formed on the lumen surface 109 (facing the elongated conveyance lumen 108 of the elongated guidewire-conveyance assembly 102). The tactile feature 110 includes a sensor device 238 (such as a capacitive sensor) mounted to (preferably, in or within) the shaft portion 201. The capacitive sensor is configured to sense (based on capacitive coupling for detection) any element that may be conductive and/or may have a dielectric difference from air or fluid in the body. The sensor device 238 is configured to detect the presence of the guidewire distal tip section 904 (of the elongated guidewire assembly 902) in response to movement (or positioning) of the guidewire distal tip section 904 toward (and proximate to) the sensor device 238. A sensor wire 240 extends from the sensor 238 toward the proximal section of the elongated medical-conveyance assembly 101. The sensor wire 240 is configured to operatively couple the sensor device 238 to a sensor controller (known and not depicted). FIG. 9 depicts the guidewire distal tip section 904 positioned at one side of the sensor device 238, and the guidewire distal tip section 904 is moved toward the sensor device 238. FIG. 10 depicts the guidewire distal tip section 904 positioned at the other side of the sensor device 238 after the sensor device 238 has made contact with the guidewire distal tip section 904 (as a result of urged movement of the guidewire distal tip section 904 past the sensor device 238, in the forward direction).

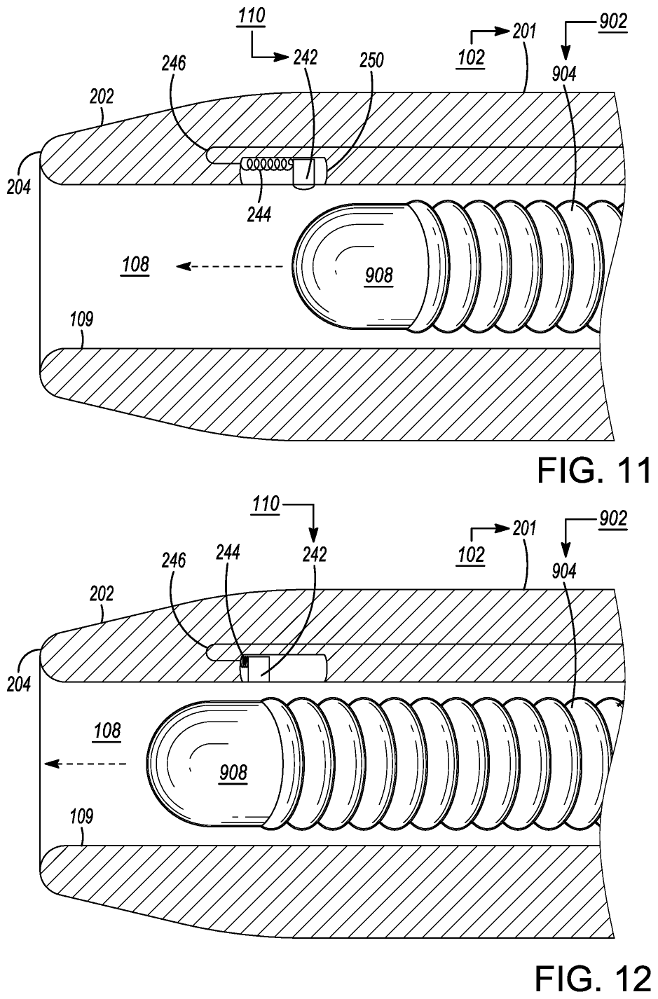

FIG. 11 and FIG. 12 depicts cross-sectional views of embodiments (implementations) of the elongated guidewire-conveyance assembly 102 and the tactile feature 110 of FIG. 1. The cross-sectional views of FIG. 11 and FIG. 12 are taken along a cross-sectional line A-A of FIG. 1.

Referring to the embodiments (implementations) as depicted in FIG. 11 and FIG. 12, the elongated guidewire-conveyance assembly 102 includes the shaft portion 201. The shaft portion 201 defines a recess feature 250 formed on the lumen surface 109 (facing the elongated conveyance lumen 108 of the elongated guidewire-conveyance assembly 102). The tactile feature 110 includes a proximity device 242 (such as, a roller element, etc. and any equivalent thereof), a biasing device 244 (such as, a spring, etc. and any equivalent thereof) and a signal wire 246. The signal wire 246 is configured to be connected to a proximity controller (known and not depicted). The signal wire 246 is configured to operatively couple the proximity device 242 to a proximity controller. The proximity device 242 is rotatably mounted to the shaft portion 201. The proximity device 242 is received, at least in part, in the recess feature 250. The biasing device 244 is configured (as depicted in FIG. 11) to normally bias (urge) the proximity device 242 toward one side of the recess feature 250 (toward the proximal end of the elongated guidewire-conveyance assembly 102). The biasing device 244 is configured (as depicted in FIG. 12) to yield and permit movement of the proximity device 242 toward the other side of the recess feature 250 (toward the distal section 104 of the elongated guidewire-conveyance assembly 102) in response to contact between the elongated guidewire-conveyance assembly 102 and the proximity device 242 with further movement of the elongated guidewire-conveyance assembly 102 toward the distal section 104. FIG. 11 depicts the guidewire distal tip section 904 positioned at one side of the proximity device 242, and the guidewire distal tip section 904 is moved toward the proximity device 242. FIG. 12 depicts the guidewire distal tip section 904 positioned at the other side of the proximity device 242 after the proximity device 242 has made contact with the guidewire distal tip section 904 (as a result of urged movement of the guidewire distal tip section 904 past the proximity device 242, in the forward direction). It will be appreciated that the proximity controller may be configured to provide sensed feedback to the user (such as haptic, audible, visual, etc.) from the proximity device 242. The proximity device 242 may be configured to detect guidewire interaction through a capacitive sensor and/or an electromagnetic sensor (preferably positioned at the guidewire distal tip section 904). The proximity controller may be configured to provide an audible click to the user. A lever arm may be tripped as the guidewire distal tip section 904 crosses through the elongated guidewire-conveyance assembly 102.

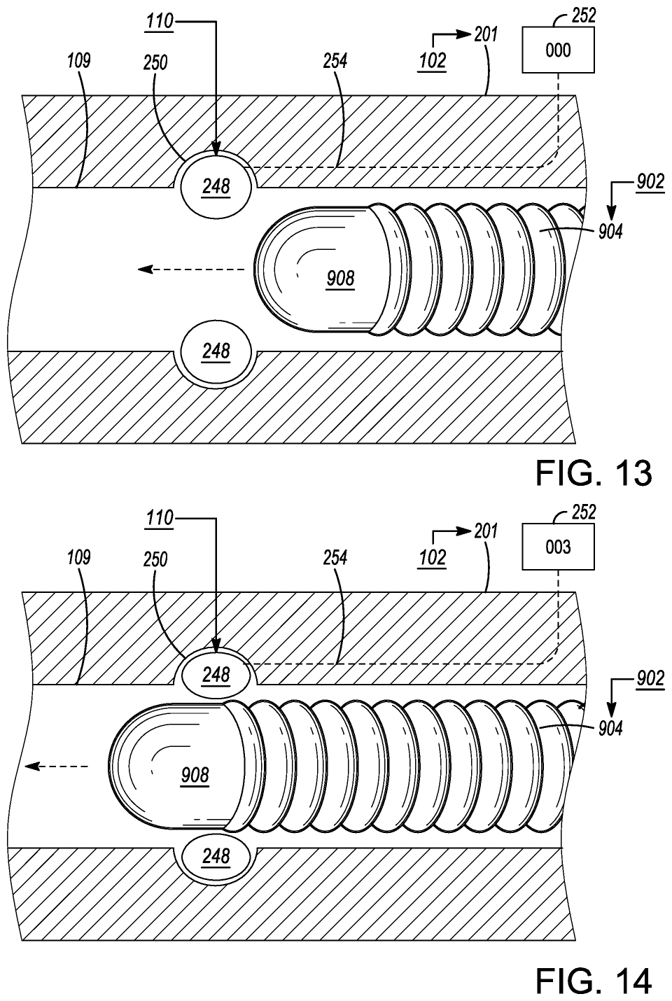

FIG. 13 and FIG. 14 depict cross-sectional views of embodiments (implementations) of the elongated guidewire-conveyance assembly 102 and the tactile feature 110 of FIG. 1. The cross-sectional views of FIG. 13 and FIG. 14 are taken along a cross-sectional line A-A of FIG. 1.

Referring to the embodiments (implementations) as depicted in FIG. 13 and FIG. 14, the elongated guidewire-conveyance assembly 102 includes the shaft portion 201. The tactile feature 110 includes a contact element 248 (such, as a roller element, etc., and any equivalent thereof). The shaft portion 201 defines a recess feature 250 formed on the lumen surface 109 (facing the elongated conveyance lumen 108 of the elongated guidewire-conveyance assembly 102). The contact element 248 is configured to be rotatably mounted to the shaft portion 201. The contact element 248 is configured to be received (at least in part) in the recess feature 250. A counter device 252 is configured to be operatively coupled (such as, wired via a counter connection 254, etc.) to the contact element 248. The counter device 252 is configured to detect, and display, the number of times the elongated guidewire assembly 902 and the contact element 248 make contact with each other in response to the elongated guidewire assembly 902 moving and making contact with the contact element 248 and moving past the contact element 248 (either in the forward direction or in the reverse direction). FIG. 13 depicts the guidewire distal tip section 904 (of the elongated guidewire assembly 902) positioned at one side of the contact element 248, and the guidewire distal tip section 904 is moved toward the contact element 248. FIG. 14 depicts the guidewire distal tip section 904 positioned at the other side of the contact element 248 after the contact element 248 has made contact with the guidewire distal tip section 904 (as a result of urged movement of the guidewire distal tip section 904 past the contact element 248, in the forward direction). The counter device 252 (as depicted in FIG. 14) indicates that the contact count has increased relative to the contact count displayed in the counter device 252 (as depicted in FIG. 13).

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus for use with an elongated medical device, the apparatus comprising:
an elongated medical-conveyance assembly defining an elongated conveyance lumen configured to receive the elongated medical device; and
a tactile feature located at the elongated conveyance lumen.

2. The apparatus of claim 1, wherein:
the elongated medical-conveyance assembly includes an elongated guidewire-conveyance assembly having a distal section and a proximal section, with the elongated conveyance lumen extending between the distal section and the proximal section, and with the elongated conveyance lumen configured to receive the elongated medical device; and
the elongated medical device includes an elongated guidewire assembly.

3. The apparatus of claim 1, wherein:
the elongated medical-conveyance assembly includes an elongated catheter assembly, an elongated dilator assembly, or an elongated sheath assembly.

4. An apparatus for use with an elongated guidewire assembly having a guidewire distal tip section and a guidewire proximal section configured to be tacitly contacted by a user, the apparatus comprising:
an elongated guidewire-conveyance assembly having a distal section and a proximal section, with an elongated conveyance lumen extending between the distal section and the proximal section, and with the elongated conveyance lumen configured to receive the guidewire distal tip section of the elongated guidewire assembly from the proximal section; and
a tactile feature located within the elongated conveyance lumen.

5. The apparatus of claim 4, wherein:
the tactile feature extends toward an interior of the elongated conveyance lumen.

6. The apparatus of claim 4, wherein:
the tactile feature is configured to tacitly interact with the guidewire distal tip section of the elongated guidewire assembly in response to contact made between the guidewire distal tip section and the tactile feature while the guidewire distal tip section is moved past the tactile feature.

7. The apparatus of claim 4, wherein:
the tactile feature is configured to tacitly interact with the guidewire distal tip section of the elongated guidewire assembly in such a way that the tactile feature, in use, imparts a tactile vibration into the guidewire distal tip section, and tactile vibration, in use, travels along the elongated guidewire assembly toward the guidewire proximal section and then to the user who is in tactile contact with the guidewire proximal section in response to tactile contact between the guidewire distal tip section and the tactile feature.

8. The apparatus of claim 4, wherein:
the tactile feature is configured to tacitly interact with the guidewire distal tip section of the elongated guidewire assembly in response to contact being made between the guidewire distal tip section and the tactile feature while the guidewire distal tip section is moved past the tactile feature.

9. The apparatus of claim 8, wherein:
the tactile feature is configured to tacitly interact with the guidewire distal tip section of the elongated guidewire assembly in such a way that the tactile feature, in use, imparts tactile vibration into the guidewire distal tip section, and tactile vibration, in use, travels along the elongated guidewire assembly toward the guidewire proximal section and then to the user who is in tactile contact with the guidewire proximal section in response to tactile contact between the guidewire distal tip section and the tactile feature.

10. The apparatus of claim 4, wherein:
the tactile feature includes a raised feature.

11. The apparatus of claim 4, wherein:
the tactile feature includes a recessed feature.

12. The apparatus of claim 4, wherein:
the tactile feature is configured to interact with the guidewire distal tip section of the elongated guidewire assembly in response to the guidewire distal tip section making physical contact with the tactile feature; and
the tactile feature is configured to provide tactile feedback to the user of the guidewire distal tip section.

13. The apparatus of claim 4, wherein:
the tactile feature includes sections of varying surface roughness formed on a lumen surface of the elongated conveyance lumen.

14. The apparatus of claim 4, wherein:
the tactile feature includes a smooth tactile lumen section and a rough tactile lumen section positioned next to the smooth tactile lumen section.

15. The apparatus of claim 4, wherein:
the tactile feature includes a helically-formed marker.

16. The apparatus of claim 4, wherein:
the tactile feature includes a surface feature formed on a lumen surface of the elongated conveyance lumen; and
the guidewire distal tip section includes a guidewire interference feature; and
the surface feature and the guidewire interference feature are configured to interact with each other.

17. The apparatus of claim 4, wherein:
the elongated guidewire-conveyance assembly includes a shaft portion; and
the shaft portion defines a recess feature formed on a lumen surface facing the elongated conveyance lumen of the elongated guidewire-conveyance assembly; and
the tactile feature includes a sensor device mounted to the shaft portion; and
the sensor device is configured to detect a presence of the guidewire distal tip section of the elongated guidewire assembly in response to movement of the guidewire distal tip section toward, and proximate to, the sensor device.

18. The apparatus of claim 4, wherein:
the elongated guidewire-conveyance assembly includes a shaft portion; and
the shaft portion defines a recess feature formed on a lumen surface facing the elongated conveyance lumen of the elongated guidewire-conveyance assembly; and
the tactile feature includes:
a proximity device; and
a biasing device; and
a signal wire configured to operatively couple the proximity device to a proximity controller; and
the proximity device is received, at least in part, in the recess feature; and
the biasing device is configured to normally bias and urge the proximity device toward one side of the recess feature and toward a proximal end of the elongated guidewire-conveyance assembly; and
the biasing device is also configured to yield and permit movement of the proximity device toward the other side of the recess feature in response to contact between the elongated guidewire-conveyance assembly and the proximity device with further movement of the elongated guidewire-conveyance assembly toward the distal section.

19. The apparatus of claim 4, wherein:
the elongated guidewire-conveyance assembly includes a shaft portion; and
the tactile feature includes a contact element; and
the shaft portion defines a recess feature formed on a lumen surface facing the elongated conveyance lumen of the elongated guidewire-conveyance assembly; and
the contact element is configured to be rotatably mounted to the shaft portion; and
the contact element is configured to be received, at least in part, in the recess feature; and
a counter device is configured to be operatively coupled to the contact element; and
the counter device is configured to detect, and display, a number of times the elongated guidewire assembly and the contact element make contact with each other in response to the elongated guidewire assembly moving and making contact with the contact element and moving past the contact element.

20. A method for using an elongated guidewire assembly having a guidewire distal tip section and a guidewire proximal section configured to be contacted by a user, the method comprising:
receiving the guidewire distal tip section of the elongated guidewire assembly into an elongated conveyance lumen of an elongated guidewire-conveyance assembly having a distal section and a proximal section, with the elongated conveyance lumen extending between the distal section and the proximal section; and
moving the guidewire distal tip section along the elongated conveyance lumen toward a tactile feature located in the elongated conveyance lumen.
